SEQUENCE LISTING (22 sequences)

SEQ ID NO: 1 - RNA, 21 nt, artificial
  oligonucleotide or target sequence
uucacaguggcuaaguucugc

SEQ ID NO: 2 - RNA, 19 nt, artificial
  oligonucleotide or target sequence
ucacaguggcuaaguucug

SEQ ID NO: 3 - RNA, 18 nt, artificial
  oligonucleotide or target sequence
ucacaguggcuaaguucu

SEQ ID NO: 4 - DNA, 18 bp, artificial
  oligonucleotide or target sequence
agaacttagccactgtga

SEQ ID NO: 5 - DNA, 18 bp, artificial
  oligonucleotide or target sequence
agaacttagccactgtga

SEQ ID NO: 6 - DNA, 18 bp, artificial
  oligonucleotide or target sequence
agaacttagccactgtga

SEQ ID NO: 7 - DNA, 18 bp, artificial
  oligonucleotide or target sequence
agaacttagccactgtga

SEQ ID NO: 8 - DNA, 18 bp, artificial
  oligonucleotide or target sequence
agaacttagccactgtga

SEQ ID NO: 9 - DNA, 18 bp, artificial
  oligonucleotide or target sequence
agaacttagccactgtga

SEQ ID NO: 10 - DNA, 18 bp, artificial
  oligonucleotide or target sequence
agaacttagccactgtga

SEQ ID NO: 11 - DNA, 18 bp, artificial
  oligonucleotide or target sequence
agaacttagccactgtga

SEQ ID NO: 12 - DNA, 18 bp, artificial
  oligonucleotide or target sequence
agaacttagccactgtga

SEQ ID NO: 13 - DNA, 18 bp, artificial
  oligonucleotide or target sequence
agaacttagccactgtga

SEQ ID NO: 14 - DNA, 18 bp, artificial
  oligonucleotide or target sequence
agaacttagccactgtga

SEQ ID NO: 15 - DNA, 18 bp, artificial
  oligonucleotide or target sequence
agaacttagccactgtga

SEQ ID NO: 16 - DNA, 18 bp, artificial
  oligonucleotide or target sequence
agaacttagccactgtga

SEQ ID NO: 17 - DNA, 19 bp, artificial
  oligonucleotide or target sequence
cagaacttagccactgtga

SEQ ID NO: 18 - DNA, 19 bp, artificial
  oligonucleotide or target sequence
cagaacttagccactgtga

SEQ ID NO: 19 - DNA, 19 bp, artificial
  oligonucleotide or target sequence
cagaacttagccactgtga

SEQ ID NO: 20 - DNA, 18 bp, artificial
  oligonucleotide or target sequence
agaacttagccactgtga

SEQ ID NO: 21 - DNA, 19 bp, artificial
  oligonucleotide or target sequence
cagaacttagccactgtga

SEQ ID NO: 22 - DNA, 18 bp, artificial
  oligonucleotide or target sequence
  misc_feature (9): n = inosine
agaacttanccactgtga

(19) United States
(12) Patent Application Publication
(10) Pub. No.: US 2024/0182889 A1
(43) Pub. Date: Jun. 6, 2024

(54) MICRORNA-27B INHIBITORS

(71) Applicant: NEUmiRNA Therapeutics Aps, København SV (DK)

(72) Inventors: Markus Sakari Kauppinen, Holte (DK); Stine Normann Hansen, København S (DK); Henrik Valdemar Klitgaard, Hellerup (DK)

(21) Appl. No.: 18/552,318

(22) PCT Filed: Mar. 28, 2022
(86) PCT No.: PCT/EP2022/058142

§ 371 Date: Sep. 25, 2023

(30) Foreign Application Priority Data
Mar. 26, 2021 (DK) PA202170146

(51) Int. Cl. C12N 15/11

(52) U.S. Cl. CPC C12N 15/11; C12N 2310/11; C12N 2310/315; C12N 2310/3231

(57) ABSTRACT

The present invention provides antisense oligonucleotides complementary to miR-27b, capable of potently inhibiting the activity of miR-27b. Such compounds are useful as pharmaceuticals for treatment of diseases in the CNS or in the PNS including neurological diseases.

### FIELD OF THE INVENTION

The present invention relates to new compounds and compositions capable of inhibiting the activity of microRNA-27b (miR-27b) in mammals such as humans. In particular, the invention provides antisense oligonucleotide compounds capable of modulating the activity of miR-27b in a human in vivo useful for treating CNS disorders, including epilepsy and memory disorders.

### BACKGROUND

Epilepsy is a serious, chronic neurological disorder characterised by recurrent spontaneous seizures affecting about 50 million people worldwide.

Present anti-epileptic drugs that are available, typically control seizures in two-thirds of patients but probably have no effect on the underlying pathophysiology. The remaining one-third of patients with epilepsy are either drug resistant or suffer from serious side effects from the presently available drugs.

An alternative to avoiding seizures in patients without the option of getting drug treatment is ketogenic diet, brain surgery, vagus nerve or intracranial stimulation.

The development of symptomatic (acquired) epilepsy is thought to involve altered expression of ion channels and neurotransmitter receptors, synaptic remodelling, inflammation, gliosis and neuronal death, among others. However, our understanding of the cellular and molecular mechanisms remains incomplete. There are currently no prophylactic treatments (“anti-epileptogenic”) following a brain injury likely to precipitate epilepsy. Similarly, there is no specific neuroprotective treatment for status epilepticus (SE), or treating acute neurologic injuries likely to cause brain damage or epilepsy, for example, stroke, or trauma.

Recent data suggest that microRNAs (miRNAs) are critical to the pathogenesis of several neurologic disorders, including epilepsy. MiRNAs comprise a class of short (˜22 nt) endogenous non-coding RNAs that mediate post-transcriptional regulation of gene expression (Ambros, Nature, 2004 Sep. 16; 431(7006):350-5/; Bartel, 2009 Jan. 23; 136(2):215-33).

Mature miRNAs serve as guide molecules for the miRISC complex by directing it to partially complementary target sites located predominantly in the 3′ untranslated regions (UTRs) of target mRNAs, resulting in translational repression and/or mRNA degradation of the targets (van Rooij & Kauppinen, EMBO Mol Med, 2014 July; 6(7):851-64). An important determinant guiding miRNA target recognition is the base pairing of the miRNA seed region (nucleotides 2-7 in the mature miRNA) with a perfectly complementary seed match site in the target mRNA 3′ UTR Bartel, 2009 Jan. 23; 136(2):215-33). MicroRNA-27b (miR-27b) has been shown to be involved in a number of neurological conditions through its regulation of activity of the Nrf2/ARE pathway. MiR-27b antagomir promoted activation of the ICH-induced Nrf2/ARE pathway and reduced the lipid peroxidation, neuroinflammation, cell death and neurological deficits otherwise seen after ICH. In PC12 cells, the miR-27b inhibitor diminished iron-induced oxidative stress, inflammation and apoptosis, and those effects were blocked by Nrf2 knockdown. These results demonstrate that miR-27b inhibition alleviates ICH-induced brain injury, which may be explained in part by its regulation of the Nrf2/ARE pathway. Induction of the Nrf2/ARE pathway has been shown to be beneficial for treatment of epilepsy.

Increased production of reactive oxygen species and oxidative stress have been implicated in the pathogenesis of numerous neurodegenerative conditions including among others Alzheimer's disease, Parkinson's disease, Huntington's disease, Friedrich's ataxia, multiple sclerosis, and stroke. The endogenous antioxidant response pathway protects cells from oxidative stress by increasing the expression of cytoprotective enzymes and is regulated by the transcription factor nuclear factor erythroid 2-related factor 2 (NRF2). In addition to regulating the expression of antioxidant genes, NRF2 has also been shown to exert anti-inflammatory effects and modulate both mitochondrial function and biogenesis. Mitochondrial dysfunction and neuroinflammation are features of many neurodegenerative diseases which underscores the potential of NRF2 as a promising therapeutic target for treatment of neurodegenerative diseases.

In summary, there is still a need for improved treatment or prevention modalities that specifically target the processes by which epilepsy and other neurological injuries likely to cause brain damage develop and that overcome some of the above-mentioned problems.

### SUMMARY OF THE INVENTION

There is a need in the market for potent antisense oligonucleotide compounds targeting miR-27b for use in treatment of diseases, where modification of miR-27b activity is beneficial. The present invention provides novel highly potent antisense oligonucleotides complementary to miR-27b, compositions, including pharmaceutical compositions comprising an effective dosage of the antisense oligonucleotides such as any one of SEQ ID NO: 5-22, and uses of such compositions for treatment of diseases where modulation of miR27b is beneficial. The said antisense oligonucleotides complementary to miR-27b, and compositions comprising such antisense oligonucleotides, including pharmaceutical compositions are potent inhibitors of miR-27b, and consequently cause upregulation of the Nrf2/ARE pathway when used in vivo. In some embodiments, the diseases treated using the compounds, compositions such as pharmaceutical compositions are diseases where upregulation of the Nrf2/ARE pathway is beneficial. In some embodiments, the disease that is treated is a disease of the CNS, such as a neurological disease.

According to an aspect, the invention concerns an antisense oligonucleotide complementary to miR-27b (SEQ ID NO 1) comprising a sequence of 18-19 nucleobases in length wherein the antisense oligonucleotides are LNA/DNA mixmers and do not contain a stretch of more than three contiguous DNA nucleotides, and wherein said antisense oligonucleotide comprises 1 and 18 phosphorothioate internucleotide linkages.

According to another aspect, the invention concerns a miR-27b inhibitory composition comprising an effective dosage of the antisense oligonucleotides complementary to miR-27b according to the invention and/or embodiments.

According to another aspect, the invention concerns a pharmaceutical composition comprising an effective dosage of the antisense oligonucleotides complementary to miR-27b according to the invention and/or embodiments and a pharmaceutically acceptable carrier.

According to another aspect, the invention concerns a pharmaceutical composition comprising the antisense oligonucleotide complementary to miR-27b according to the invention and/or embodiments, wherein said antisense oligonucleotide complementary to miR-27b is the sole active pharmaceutical ingredient.

According to another aspect, the invention concerns the use of the antisense oligonucleotides complementary to miR-27b according to the invention, such as anyone of SEQ ID NO: 5-22 for use as a medicament.

In a preferred embodiment, the antisense oligonucleotide according to the invention comprises SEQ ID NO 8.

In another preferred embodiment, the antisense oligonucleotide according to the invention comprises SEQ ID NO 12.

In another preferred embodiment, the antisense oligonucleotide according to the invention comprises SEQ ID NO 16.

In another preferred embodiment, the antisense oligonucleotide according to the invention comprises SEQ ID NO 19.

In another preferred embodiment, the antisense oligonucleotide according to the invention comprises SEQ ID NO 20.

In another preferred embodiment, the antisense oligonucleotide according to the invention comprises SEQ ID NO 22.

According to another aspect, the invention concerns a method for the treatment of the diseases according to the invention and/or embodiments by use of the antisense oligonucleotides complementary to miR-27b according to the invention and/or embodiments or the composition according to the invention and/or embodiments.

According to another aspect, the invention concerns a method of diagnosing a disease according to the invention and/or embodiments by use of the antisense oligonucleotides complementary to miR-27b according to the invention and/or embodiments or the composition according to the invention and/or embodiments.

There is a need for the compounds of the invention, as many of the aforementioned diseases cannot be treated in a sufficient manner, and/or where presently available treatments cause serious side effects.

### DETAILED DESCRIPTION OF THE INVENTION

In describing the embodiments of the invention, specific terminology will be resorted to for the sake of clarity. However, the invention is not intended to be limited to the specific terms so selected, and it is understood that each specific term includes all technical equivalents, which operate in a similar manner to accomplish a similar purpose.

The term “therapeutically effective amount”, or “effective amount” or effective dose”, refers to an amount of a therapeutic agent, which confers a desired therapeutic effect on an individual in need of the agent. The effective amount may vary among individuals depending on the health and physical condition of the individual to be treated, the taxonomic group of the individuals to be treated, the formulation of the composition, the method of administration, assessment of the individual's medical condition, and other relevant factors.

The term “treatment” refers to any administration of a therapeutic medicament, herein comprising an antisense oligonucleotide that partially or completely cures or reduces one or more symptoms or features of a given disease.

The term “compound” as used herein, refers to a compound comprising an anti miR-27b oligonucleotide according to the invention. In some embodiments, a compound may comprise other elements a part from the oligonucleotide of the invention. Such other elements may in non-limiting example be a delivery vehicle which is conjugated or in other way bound to the oligonucleotide.

“Antisense oligonucleotide” means a single-stranded oligonucleotide having a nucleobase sequence that permits hybridization to a corresponding region or segment of a target nucleic acid. The antisense oligonucleotide of the present invention is preferably a “mixmer”.

A “mixmer” is an antisense oligonucleotide, comprising a mix of nucleoside analogues such as LNA and DNA nucleosides (LNA/DNA mixmer), and wherein the antisense oligonucleotide does not comprise an internal region having a plurality of nucleosides (such as a region of at least 6 or 7 DNA nucleotides), capable of recruiting an RNAse, such as RNAseH, wherein the nucleosides comprising the internal region are chemically distinct from the nucleoside or nucleosides comprising the external wings.

“Nucleoside analogues” are described by e.g. Freier & Altmann; Nucl. Acid. Res., 1997, 25, 4429-4443 and Uhlmann; Curr. Opinion in Drug Development, 2000, 3(2), 293-213, and examples of suitable and preferred nucleoside analogues are provided by WO2007031091, which are hereby incorporated by reference.

“5-methylcytosine” means a cytosine modified with a methyl group attached to the 5′ position. A 5-methylcytosine is a modified nucleobase.

“2′-O-methoxyethyl” (also 2′-MOE and 2′-O(CH˜)˜—OCH3) refers to an O-methoxy-ethyl modification at the 2′ position of a furanose ring.

“2′-MOE nucleoside” (also 2′-O-methoxyethyl nucleoside) means a nucleoside comprising a 2′-MOE modified sugar moiety.

A “locked nucleic acid” or “LNA” is often referred to as inaccessible RNA, and is a modified RNA nucleobase. The ribose moiety of an LNA nucleobase is modified with an extra bridge connecting the 2′ oxygen and 4′ carbon. An LNA oligonucleotide offers substantially increased affinity for its complementary strand, compared to traditional DNA or RNA oligonucleotides. In some aspects bicyclic nucleoside analogues are LNA nucleotides, and these terms may therefore be used interchangeably, and in such embodiments, both are characterized by the presence of a linker group (such as a bridge) between C2′ and C4′ of the ribose sugar ring. When used in the present context, the terms “LNA unit”, “LNA monomer”, “LNA residue”, “locked nucleic acid unit”, “locked nucleic acid monomer” or “locked nucleic acid residue”, refer to a bicyclic nucleoside analogue. LNA units are described in inter alia WO 99/14226, WO 00/56746, WO 00/56748, WO 01/25248, WO 02/28875, WO 03/006475, WO2015071388, and WO 03/095467.

“Beta-D-Oxy LNA”, is a preferred LNA variant.

“Bicyclic nucleic acid” or “BNA” or “BNA nucleosides” mean nucleic acid monomers having a bridge connecting two carbon atoms between the 4′ and 2′ position of the nucleoside sugar unit, thereby forming a bicyclic sugar. Examples of such bicyclic sugar include, but are not limited to A) pt-L-methyleneoxy (4′-CH2-O-2′) LNA, (B) P-D-Methyleneoxy (4′-CH2-O-2′) LNA, (C) Ethyleneoxy (4′-(CH2)2-O-2′) LNA, (D) Aminooxy (4′-CH2-O—N(R)-2′) LNA and (E) Oxyamino (4′-CH2-N(R)—O-2′) LNA.

As used herein, LNA nucleotides include, but are not limited to, nucleotides having at least one bridge between the 4′ and the 2′ position of the sugar wherein each of the bridges independently comprises 1 or from 2 to 4 linked groups independently selected from —[C(R˜)(R2)]ₙ-, —C(R˜)═C(R2)-, —C(R˜)═N, —C(═NREM)-, —C(═O)—, —C(═S)—, —O—, —Si(Ri)q-, —S(═O)— and —N(R&)-; wherein: x is 0, 1, or 2; n is 1, 2, 3, or 4; each R& and R2 is, independently, H, a protecting group, hydroxyl, C»C» alkyl, substituted C»(—CHz-) group connecting the 2′ oxygen atom and the 4′ carbon atom, for which the term methyleneoxy (4′-CH&—O-2′) LNA is used.

Furthermore; in the case of the bicyclic sugar moiety having an ethylene bridging group in this position, the ethyleneoxy (4′-CH&CH&—O-2′) LNA is used. n-L-methyleneoxy (4′-CH&—O-2′), an isomer of methyleneoxy (4′-CH&—O-2′) LNA is also encompassed within the definition of LNA, as used herein.

In some embodiments, the nucleoside unit is an LNA unit selected from the list of beta-D-oxy-LNA, alpha-Loxy-LNA, beta-D-amino-LNA, alpha-L-amino-LNA, beta-D-thio-LNA, alpha-L-thio-LNA, 5′-methyl-LNA, beta-D-ENA and alpha-L-ENA.

“cEt” or “constrained ethyl” means a bicyclic sugar moiety comprising a bridge connecting the 4′-carbon and the 2′-carbon, wherein the bridge has the formula: 4′-CH(CHq)-O-2′.

“Constrained ethyl nucleoside” (also cEt nucleoside) means a nucleoside comprising a bicyclic sugar moiety comprising a 4′-CH(CH3)-O-2′ bridge. cEt and some of its properties are described in Pallan et al. Chem Commun (Camb). 2012, August 25; 48(66): 8195-8197.

“Tricyclo (tc)-DNA” belongs to the class of conformationally constrained DNA analogs that show enhanced binding properties to DNA and RNA. Structure and method of production may be seen in Renneberg et al. Nucleic Acids Res. 2002 Jul. 1; 30(13): 2751-2757.

“2′-fluoro”, as referred to herein is a nucleoside comprising a fluoro group at the 2′ position of the sugar ring. 2′-fluorinated nucleotides are described in Peng et al. J Fluor Chem. 2008 September; 129(9): 743-766.

“2′-O-methyl”, as referred to herein, is a nucleoside comprising a sugar comprising an —OCH3 group at the 2′ position of the sugar ring.

“Conformationally Restricted Nucleosides (CRN)” and methods for their synthesis, as referred to herein, are described in WO2013036868, which is hereby incorporated by reference. CRN are sugar-modified nucleosides, in which, similar to LNA, a chemical bridge connects the C2′ and C4′ carbons of the ribose. However, in a CRN, the C2′-C4′ bridge is one carbon longer than in an LNA molecule. The chemical bridge in the ribose of a CRN locks the ribose in a fixed position, which in turn restricts the flexibility of the nucleobase and phosphate group. CRN substitution within an RNA- or DNA-based oligonucleotide has the advantages of increased hybridization affinity and enhanced resistance to nuclease degradation.

“Unlocked Nucleic Acid” or “UNA”, is as referred to herein unlocked nucleic acid typically where the C2-C3 C—C bond of the ribose has been removed, forming an unlocked “sugar” residue (see Fluiter et al., Mol. Biosyst., 2009, 10, 1039, hereby incorporated by reference, and Snead et al. Molecular Therapy-Nucleic Acids (2013) 2, e103;).

“Target region” means a portion of a target nucleic acid to which one or more antisense compounds is targeted.

“Targeted delivery” as used herein means delivery, wherein the antisense oligonucleotide has either been formulated in a way that will facilitate efficient delivery in specific tissues or cells, or wherein the antisense oligonucleotide in other ways has been for example modified to comprise a targeting moiety, or in other way has been modified in order to facilitate uptake in specific target cells.

### Compounds

The antisense oligonucleotides of the invention are designed to target microRNA-27b (miR-27b) Specific antisense oligonucleotides have been designed to target regions of miR-27b having the mature sequence 5′ uucacaguggcuaaguucugc 3′ (SEQ ID NO: 1) (miRBase acc #MIMAT0000419).

The above reference to “miRBase” is according to miRBase release 22.1.

The term “miR-27b related neurological disease” as used herein means diseases where disease pathology is linked with upregulation of miR-27b activity, or where downregulation of miR-27b activity will be beneficial for treatment of the disease.

In some embodiments, the invention provides antisense oligonucleotides designed to target part of or the whole of 5′ ucacaguggcuaaguucug 3′ (SEQ ID NO: 2).

In some embodiments, the antisense oligonucleotides of the invention are designed to target at least 5′ ucacaguggcuaaguucu 3′ (SEQ ID NO: 3).

In some embodiments, the antisense oligonucleotides comprise the sequence 5′agaacttagccactgtga3′ (SEQ ID NO: 4).

In some embodiments, the antisense oligonucleotide is 18 or 19 nucleotides in length, and comprises the sequence 5′ agaacttagccactgtga 3′ (SEQ ID NO: 4).

In some embodiments, the antisense oligonucleotide is 18 or 19 nucleotides in length, comprises the sequence 5′ agaacttagccactgtga 3′ (SEQ ID NO: 4) and is a mixmer.

In some embodiments, the antisense oligonucleotide targeting miR-27b is 18 or 19 nucleotides in length, comprises the sequence 5′ agaacttagccactgtga 3′ (SEQ ID NO: 4) and is an LNA/DNA mixmer. It has surprisingly been found that antisense oligonucleotides which are LNA/DNA mixmers, 18 or 19 nucleotides in length, and comprise SEQ ID NO: 4 are particularly potent in downregulating miR-27b activity.

Such antisense oligonucleotides complementary to miR-27b show superior efficiency in downregulating their target miR-27b when they are 18 or 19 nucleotides in length, LNA/DNA mixmers, comprising from 50-70% LNA and has no more than three consecutive DNA nucleotides. In some embodiments, the invention provides an antisense oligonucleotide complementary to miR-27b consisting of a sequence of 18-19 nucleobases in length that is a mixmer which does not comprise a region of more than three consecutive DNA nucleotides, and which comprises between seven and 14 affinity-enhancing nucleotide analogues, and wherein the antisense oligonucleotide comprises between 1 and 18 phosphorothioate internucleotide linkages, and wherein the oligonucleotide is complementary to any of SEQ ID NO: 2-3, or which comprises SEQ ID NO: 4. In some embodiments, the antisense oligonucleotide complementary to miR-27b, is 18 or 19 nucleotides in length, comprises SEQ ID NO: 4 and is an LNA/DNA mixmer having between 50 and 70% LNA, such as between 52 and 68% LNA, such as at least 50% LNA, such as at least 52% LNA.

In some embodiments, the antisense oligonucleotides complementary to miR-27b according to any one of the above embodiments, have two terminal LNA nucleotides in each end. Further, in preferred embodiments, the LNA used in the antisense oligonucleotides of the invention are Beta-D-Oxy LNA.

In some preferred embodiments, all LNA cytosines are 5-methylcytosine, i.e. in sequence listings, all Capital C's are methyl C's.

For in vivo use stability of the antisense oligonucleotide will benefit from having one or more phosphorothioate linkages. In some embodiments, the antisense oligonucleotides complementary to miR-27b comprise phosphorothioate internucleoside bonds, such as at least one bond is phosphorothioate, or in some instances, the oligonucleotides have a complete phosphorothioate backbone, i.e. all internucleoside linkages are phosphorothioate linkages.

The inventors have identified a series of highly potent antisense oligonucleotides complementary to miR-27b that all have the features listed in the above embodiments. These compounds are listed in Table 1 as SEQ ID NO's: 5-22. All of these compounds are preferred. In some embodiments, the compounds having any one of SEQ ID NO's: 8, 12, 16, 19, 20 and 22 are especially preferred.

Table 1 describes SEQ ID NO: 5-22 which are LNA/DNA mixmers that are antisense oligonucleotides complementary to miR-27b. In all sequences of Table 1, Capital C is methyl-C(5-methylcytosine).

TABLE 1
AntimiR-27b compounds

| SEQ | | | # | % |
|---|---|---|---|---|
| ID NO | | Length | LNA | LNA |
| 5 | AGaaCTtAgCCaCtGtGA | 18 | 11 | 61 |
| 6 | AGaAcTTaGcCACtGtGA | 18 | 12 | 67 |
| 7 | AGaaCTTaGCcaCtGtGA | 18 | 11 | 61 |
| 8 | AGaActTAgcCaCTGtGA | 18 | 11 | 61 |
| 9 | AGaaCTtAGcCaCTgTGA | 18 | 12 | 67 |
| 10 | AGaaCTtAgCCAcTgTGA | 18 | 12 | 67 |
| 11 | AGaAcTTaGcCACtgTGA | 18 | 12 | 67 |
| 12 | AGaaCTtAGcCaCtgTGA | 18 | 11 | 61 |
| 13 | AGAacTTagCcACTgtGA | 18 | 11 | 61 |
| 14 | AGaaCTtaGccAcTgTGA | 18 | 10 | 55 |
| 15 | AGaActTaGccACTgTGA | 18 | 11 | 61 |
| 16 | AGaActTagCcaCTgTGA | 18 | 10 | 55 |
| 17 | CAgaAcTTagCcaCTgTGA | 19 | 11 | 58 |
| 18 | CAGaACtTAgcCaCTGtGA | 19 | 13 | 68 |
| 19 | CAgaaCTtaGccACtgTGA | 19 | 10 | 52 |
| 20 | AGAacTTagCcACTgtGA | 18 | 11 | 61 |
| 21 | CAGaACtTAgcCaCTGtGA | 19 | 13 | 68 |
| 22 | AGAacTTaiCcACTgtGA | 18 | 11 | 61 |

In table 1, upper case letters indicate LNA and lower case letters are DNA.
The letter “i” is inosine.
Capital C is LNA 5-methylcytocine.
All internucleoside bonds are phosphorothioate bonds.

In some instances, it will add to the potency or other characteristics of a compound to replace one or more of the DNA nucleotides of an LNA/DNA mixmer comprising other affinity enhancing nucleotides than LNA.

In some instances, the antisense oligonucleotides complementary to miR-27b of the invention are LNA/DNA mixmers wherein one or more DNA nucleotides have been replaced with one or more nucleosides that are anyone of tricyclo-DNA, 2′-Fluoro, 2′-O-methyl, 2′methoxyethyl (2′MOE), 2′ cyclic ethyl (cET), UNA, 2′fluoro and Conformationally Restricted Nucleoside (CRN).

### Compositions and Uses

The antisense oligonucleotide complementary to miR-27b of the present invention are well suited for use as a medicament. Further, miR-27 inhibitory compositions comprising the antisense oligonucleotide complementary to miR-27b of the invention are provided. Such compositions may be used for inducing the Nrf-2/ARE pathway in a mammal, such as in a human. In some preferred embodiments, the antisense oligonucleotide complementary to miR-27b for use as a medicament, or the antisense oligonucleotide complementary to miR-27b comprised in an inhibitory composition is anyone of SEQ ID NO's: 5-22).

The antisense oligonucleotide complementary to miR-27b and compositions of the invention show great potential in medical use, such as for the treatment, alleviation, pre-emptive treatment or prophylaxis of a miR-27b related disease where modification of miR-27b activity, or induction of the Nrf-2/ARE pathway is beneficial. A number of such diseases have been identified, including diseases of the CNS or PNS. Consequently, in some embodiments the anti miR-27b oligonucleotides of the invention are for treatment, alleviation, pre-emptive treatment or prophylaxis of a miR-27b related disease of the CNS or PNS.

In some embodiments, such CNS or PNS disorders include neurological disorders, neurodegenerative disorders or neurodevelopmental disorders, and therefore, the anti miR-27b compounds of the invention in some embodiments are for treatment, alleviation, pre-emptive treatment or prophylaxis of a neurological disorder, a neurodegenerative disorder, a neurodevelopmental disorder, a genetic disorder and/or a genetic neurodevelopmental disorder.

It has been shown that induction of the Nrf2/Are pathway is beneficial for treatment of neurological disorders such as epilepsy, or various states of epilepsy. In some embodiments, the compounds of the invention are for use in treatment, alleviation, pre-emptive treatment or prophylaxis of epilepsy, such as drug resistant epilepsy or seizures in epilepsy or spontaneous seizures in epilepsy or therapy resistant seizures. In some embodiments, the epilepsy is a focal epilepsy, preferably wherein said focal epilepsy is focused in the frontal lobe, the parietal lobe, the occipital lobe or the temporal lobe. In some embodiments, the epilepsy is a generalised epilepsy, preferably wherein said generalised epilepsy is selected among absences, myoclonic seizures, tonic-clonic seizures, tonic seizures, atonic seizures, clonic seizures and spasms. In some embodiments, the epilepsy is status epilepticus. In some embodiments, the epilepsy is selected among autosomal dominant nocturnal frontal lobe epilepsy, continuous spike-and-waves during slow sleep, Dravet syndrome, epilepsy developed after apoplexy, epileptic encephalopathy, Gelastic epilepsy, absences, benign neonatal seizures, Jeavons syndrome, Juvenile myoclonic epilepsy, Landau-Kleffner Syndrom, Lennox-Gastaut syndrome, Mesial temporal lobe epilepsy, myoclonic astatic epilepsy, Ohtahara Syndrom, Panayiotopoulos syndrome, PCDH19 syndrom, benign childhood epilepsy with centrotemporal spikes, Sturge-Weber syndrome, symptomatic focal epilepsy, transient epileptic amnesia and West syndrome.

In some embodiments, the compounds of the invention such as any of SEQ ID NO: 5-22 are for prevention or prophylaxis or alleviation or treatment of epilepsy together with a comorbidity selected among a psychiatric disorder, a cognitive disorder, a sleep disorder, a cardiovascular disorder, a respiratory disorder, an inflammatory disorder, a psychiatric disorder, anxiety, pain, cognitive impairment, depression, dementia, headache, migraine, heart disease, ulcers, peptic ulcers, arthritis and osteoporosis.

Evidence of neuroprotective effects of miR-27b inhibition and of stimulation of the Nrf2/ARE pathway exist, and thus the present compounds and compositions comprising effective dosages of those compounds are for use in prevention or prophylaxis or pre-emptive treatment or alleviation or treatment of neuronal damage such as hippocampal damage.

In some embodiments, the compounds and compositions of the invention is for treatment, alleviation, pre-emptive treatment or prophylaxis of oxidative stress, inflammation and/or apoptosis.

In some instances the compounds and compositions are for use for treatment, alleviation, pre-emptive treatment or prophylaxis of intracerebral hemorrhage-induced brain injury, ischemic stroke, hemorrhagic stroke or stroke.

In some embodiments, the compounds according to the invention are for use in the treatment, alleviation, amelioration, pre-emptive treatment or prophylaxis of an autoimmune disease, a memory disorder, hippocampal sclerosis, Parkinsons Disease, a demyelinating disease, multiple sclerosis, spinal cord injury, acute spinal cord injury, amyotrophic lateral sclerosis, Progressive bulbar palsy, Progressive muscular atrophy, Primary lateral sclerosis, ataxia, bell's palsy, a hereditary neurological disease, Charcot-Marie-Tooth, a headache, Horton's headache, migraine, pick's disease, progressive supranuclear palsy, multi-system degeneration, motor neuron diseases, Huntington's disease, prion disease, Creutzfeldt-Jakob disease, corticobasal degeneration, aphasia, primary progressive aphasia or symptoms or effects thereof.

Nrf2 is ubiquitously expressed in the CNS, and activate neuroprotective processes relevant for neurological disease states. Accordingly, the compounds of the invention are capable of acting as neuroprotective drugs through their inhibition of miR-27b and subsequent upregulation of Nrf2, and stimulation of the Nrf2/ARE pathway.

Therefore, in some embodiments, the compounds of the invention are for use in treatment, alleviation, amelioration, pre-emptive treatment or prophylaxis of dementia, such as dementia selected among Alzheimer disease, vascular dementia, frontotemporal dementia and Lewy bodies dementia.

In some embodiments the compounds according to the invention are for use in the treatment, alleviation, amelioration, pre-emptive treatment or prophylaxis of pain, such as pain associated with osteoarthritis.

In some embodiments, the compounds according to the invention are for use in the treatment, alleviation, amelioration, pre-emptive treatment or prophylaxis of a psychiatric disease wherein modulation of miR-27b activity is beneficial, such as any of schizophrenia, depression, bipolar disorder, attention deficit hyperactivity disorder, autism, anxiety or Tourette.

miR-27b has been shown to be involved in the pathology of certain cancers, such as in the angiogenesis process, and in some embodiments, the compounds of the invention are for use in the treatment, alleviation, amelioration, pre-emptive treatment or prophylaxis of an angiogenesis related disease.

In some embodiments, the compounds of the invention are for use in the treatment alleviation, amelioration, pre-emptive treatment or prophylaxis of a cancer, such as in non-limiting example any one of a cancer of the central nervous system, glioma, cancer in the skin, melanoma, head or neck cancer, squamous cell carcinoma, preferably tongue squamous cell carcinoma or oral squamous cell carcinoma, a hematologic cancer, preferably myeloma or lymphoma, more preferably diffuse large B-cell lymphoma, a breast cancer, triple negative breast cancer, a thyroid cancer, anaplastic thyroid cancer, a liver cancer, hepatocellular carcinoma, a cancer selected from the group of gastric cancer, cervical cancer, endometrial cancer, hemangioma, lung cancer, pancreatic cancer, bladder cancer, prostate cancer and colorectal cancer, such as migration and invasion in colorectal cancer. In some embodiments, the compounds of the invention are for use in the treatment, alleviation, amelioration, pre-emptive treatment or prophylaxis of cancer metastasis. Prader-Willis Syndrome and Anglemans syndrome and arthritis conditions have immunoinflammatory traits. Therefore, in some embodiments, the compounds according to the invention are for use in the treatment, alleviation, amelioration, pre-emptive treatment or prophylaxis of Prader-Willis Syndrome or Anglemans Syndrome, arthritis, osteoarthritis miR-27b is overexpressed in certain cardiac conditions, and is involved in the development of heart failure. In some embodiments the compounds of the invention are for use in the treatment, alleviation, amelioration, pre-emptive treatment or prophylaxis of a cardiovascular disorder, including but not limited to any one of atherosclerosis, peripheral artery disease, postoperative atrial fibrillation, heart failure and chronic heart failure, intracerebral haemorrhage-induced brain injury or stroke.

miR-27b expression has been shown to be involved in liver conditions, including development of nonalcoholic fatty liver disease. The compounds of the invention are for use in the treatment, alleviation, pre-emptive treatment or prophylaxis of a liver disorder. In some embodiments, the liver disorder is selected among non-alcoholic fatty liver, fatty liver, fatty liver fibrosis, liver fibrosis and hepatoma.

Pulmonary sarcoidosis is characterised in that miR-27b is upregulated in PB lymphocytes of patients. In some embodiments, the compounds of the invention are for the treatment, alleviation, amelioration, pre-emptive treatment or prophylaxis of an autoimmune disease, a granulomatous disease, a connective tissue disease or sarcoidosis, or a pulmonary disorder. In some embodiments, the pulmonary disorder is pulmonary sarcoidosis.

In some embodiments, the compounds of the invention are for use in the treatment, alleviation, amelioration, pre-emptive treatment or prophylaxis of an infection. In some embodiments, the infection treated, alleviated, ameliorated, pre-emptively treated or prophylactically treated is any of sepsis, meningitis and encephalitis. In some embodiments, the compounds according to the invention are for the treatment, alleviation, amelioration, pre-emptive treatment or prophylaxis of a viral infection, including any of a herpes virus infection, a human papilloma virus infection, a Cytomegalovirus infection, or a herpes simplex virus infection.

MiR-27b has been shown to be implicated in angiogenesis and in the development of retinal disease, including age related macular degeneration. In some embodiments, the compounds of the invention are for use in the treatment, alleviation, amelioration, pre-emptive treatment or prophylaxis of a disorder of the retina, such as any one from the list of retinopathy, diabetic retinopathy and age-related macular degeneration (AMD).

miR-27b is involved in development of insulin resistance and glucose metabolism, and is consequently a target for treatment of metabolic disorders, such as diabetes. In some embodiments, the compounds of the invention are for use in the treatment, alleviation, amelioration, pre-emptive treatment or prophylaxis of a metabolic disorder, such as diabetes or type 2 diabetes.

miR-27b is involved in the development of neurofibromatosis by targeting NF1. In some embodiments, the compounds of the invention are for use in the treatment, alleviation, amelioration, pre-emptive treatment or prophylaxis of neurofibromatosis, including neurofibromatosis type 1.

The compounds of the invention are potent inhibitors of miR-27b, and will in effective dosages be valuable medicaments for use in the treatment, alleviation, amelioration, pre-emptive treatment or prophylaxis of the diseases described above. In some instances, combination with other active pharmaceutical compounds may provide a better effect, such as an improved effect, such as an additive effect or a synergistic effect.

In some embodiments, the anti miR-27b oligonucleotide compounds of the invention, such as any one of SEQ ID NO: 5-22 are for use in combination with another pharmaceutical compound, for use in the treatment, alleviation, amelioration, pre-emptive treatment or prophylaxis of any one of the diseases mentioned above, including but not limited to neurological and psychiatric disorders. In some embodiments the antisense oligonucleotide complementary to miR-134 of the invention is for use in combination with one or more other therapies for the diseases mentioned in the embodiments, such as for treatment of neurological and psychiatric disorders. In some embodiments, the anti miR-27b oligonucleotide compounds of the invention, such as any one of SEQ ID NO: 5-22 are for use in combination with a miR-134 inhibitor or an adenosine kinase inhibitor or both, for use in the treatment, alleviation, amelioration, pre-emptive treatment or prophylaxis of any one of the diseases mentioned above,

In some embodiments, the therapy using the compounds according to the present invention induce the Nrf2/ARE pathway in a mammal, such as in a human. In some embodiments, the compounds, uses, treatment, alleviation, amelioration, pre-emptive treatment or prophylaxis is in a mammal, such as a human.

In some embodiments, a pharmaceutical composition comprising the anti miR-27b oligonucleotide compound as the sole active pharmaceutical ingredient is provided. In some embodiments, the pharmaceutical composition comprises the anti miR-27b compound and a pharmaceutically acceptable carrier.

Administration of pharmaceuticals in the most optimal manner is important, in order to make available the active ingredient to the target tissue in an effective dosage. miR-27b is a relevant target for diseases of the CNS, PNS and peripheral organs. Consequently, the method of administrating the anti miR-27b compounds must be selected according to the disease to be treated. Many options for administration methods of drugs are available, including those described in the below embodiment. In some embodiments, the compositions such as the pharmaceutical compositions comprising the anti miR-27b compounds of the invention are for administration by any one of subcutaneous administration, intravenous administration, parenteral administration, nasal administration, pulmonary administration, rectal administration, vaginal administration, intrauterine administration, Intraurethral administration, administration to the eye, administration to the ear, cutaneous administration, intradermal administration, intramuscular administration, intraperitoneal administration, epidural administration, intraventricular administration, intracerebral, intrathecal administration or oral administration or for administration directly into the brain or cerebrospinal fluid, or wherein said composition is administered as an implant.

In some embodiments, the pharmaceutical composition of the invention is for administration in a pump, preferably wherein said pump is a mini-osmotic pump.

In some embodiments, the pharmaceutical composition of the invention is for intraventricular administration facilitated by an intraventricular catheter, preferably wherein said catheter is attached to a reservoir, preferably wherein said reservoir is an Ommaya reservoir.

The pharmaceutical compositions according to the invention are for administration in effective dosages which may be maintained by subsequent administrations wherein said composition is administrated with an interval of 1 day, 2 days, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or preferably 120 days.

In some embodiments, the pharmaceutical composition according to the invention is administrated with an interval of between 1-200 days, 10-190 days, 20-180 days, 30-170 days, 40-160 days, 50-150 days, 60-140 days, 70-130 days, 80-120 days, 90-110 days or preferably about 100 days.

The antisense oligonucleotide complementary to miR-27b are useful in methods of treatment of the diseases described above. In some embodiments, the antisense oligonucleotides of SEQ ID NO: 5-22 are for use in methods of treatment of the above listed diseases, including for treatment of the CNS or PNS diseases listed above.

The anti miR-27b compounds are in some instances comprised in compositions, pharmaceutical compositions for use in the treatment, pre-emptive treatment, amelioration, alleviation or prophylaxis of the diseases described above, and wherein the treatment is anyone of preventive, curative or disease modifying.

In some of the diseases described above, miR-27 is overexpressed compared to non diseased persons. In such instances the antisense oligonucleotide complementary to miR-27b of the invention are for use in a method of diagnosing the disease.

### Dosages

The expression “effective dosage” denotes the dose of a drug that will achieve the desired effect.

In the context of the present invention, the desired effect is lowering of the activity of miR-27b.

Lowering of the activity of miR-27b can be measured by either measuring the level of miR-27b, for example when using oligonucleotides which result in degradation of miR-27b or miR-27b precursors, or may be measured by measuring the derepression of microRNA-27b targets (such as mRNAs which comprise a miR-27b binding site and whose expression is regulated by miR-27b (miR-27b target mRNAs)). In some embodiments the efficacy of treatment is measured by measuring the upregulation of Nrf2. miR-27b inhibition may therefore be measured directly or indirectly via secondary indicators of miR-27b activity.

The compounds of the invention are for use in effective dosages, and the compositions comprise effective dosages of the compounds of the invention.

In some embodiments, the dosage of the compound administered at each dosing, such as unit dose, is within the range of 0.0001 mg/kg-25 mg/kg.

In some embodiments, the effective dose is a dose that is sufficient to down-regulate miR-134 or the activity thereof, to a significant level over the time period between successive administration dosages, such as a level which is a therapeutic benefit to the subject.

The pharmaceutical compositions of the invention may in some embodiments be made for administration to provide for an initial dosage build up phase, which may, depending on the disease pathology, be followed by a maintenance dosage scheme for the purpose of maintaining a concentration of the compound in the subject, such as in a target tissue of the subject, which will be effective in the treatment of the disease. The effectiveness of the dosages may in example be measured by observation of a disease parameter indicative of the state of the disease, or may depending on the target tissue, be measurable by observation of various tissue parameters, such as activity of a miR-27b target RNA, or in alternative example on a measurable disease state dependent parameter in plasma.

### Drug Delivery

Various delivery systems are known and can be used to administer a therapeutic of the invention. Methods of administration includes but are not limited to subcutaneous administration, intravenous administration, parenteral administration, nasal administration, pulmonary administration, rectal administration, vaginal administration, intrauterine administration, Intraurethral administration, administration to the eye, administration to the ear, cutaneous administration, intradermal administration, intramuscular administration, intraperitoneal administration, epidural administration, intraventricular administration, intracerebral, intrathecal administration or oral administration or administration directly into the brain or cerebrospinal fluid. The compositions may be administered by any convenient route, for example by infusion or bolus injection, by absorption through epithelial or mucocutaneous tissue (e.g., oral mucosa, rectal and intestinal mucosa, etc.) and may be administered together with or without other biologically active agents. Administration can be systemic or local. In addition, it may be desirable to administer the compositions of the invention into the central nervous system by any suitable route, including intraventricular and intrathecal administration. Intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. Preferably, the therapeutic is delivered to the CNS or PNS.

Delivery means include inhaled delivery, intramuscular delivery directly into a muscle by syringe or mini osmotic pump, intraperitoneal administration directly administered to the peritoneum by syringe or mini osmotic pump, subcutaneous administration directly administered below the skin by syringe, intraventricular administration direct administration to the ventricles in the brain, by injection or using small catheter attached to an osmotic pump. Further, an implant can be prepared (e.g. small silicon implant) that will be placed in a muscles or directly onto the spinal cord. It may be desirable to administer the compositions of the invention locally to the area in need of treatment; this may be achieved for example and not by way of limitation, by topical application, by injection, by means of a catheter, by means of a suppository, or by means of an implant, said implant may be of a porous, non-porous, or gelatinous material, including membranes, such as sialastic membranes, or fibers.

### Pharmaceutical Compositions

The present invention also provides pharmaceutical compositions. Such compositions may comprise a therapeutically effective amount of the therapeutic, and a pharmaceutically acceptable carrier. The term “pharmaceutically acceptable” may be defined as approved by a regulatory agency. The regulatory agency may for example be the European Medicines Agency, a Federal or a state government or listed in the U.S. Pharmacopeia or other generally recognized pharmacopeia for use in animals, and more particularly in humans. The term “therapeutically effective amount” may be defined as an amount of therapeutic which results in a clinically significant inhibition, amelioration or reversal of development or occurrence of a disorder or disease. The term “carrier” may refer to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water may be a preferred carrier when the pharmaceutical composition is administered intravenously. Saline solutions and aqueous dextrose and glycerol solutions may also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, may also contain wetting or emulsifying agents, or pH buffering agents. These compositions may take the form of solutions, suspensions, emulsion, tablets, pills, capsules, powders, sustained-release formulations and the like. The composition may be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. Such compositions may contain a therapeutically effective amount of the therapeutic, preferably in purified form, together with a suitable amount of carrier so as to provide the form for proper administration to the patient. The formulation may suit the mode of administration. Compositions for intravenous administration may be solutions in sterile isotonic aqueous buffer. Where necessary, the composition may also include a solubilizing agent and a local anaesthetic such as lignocaine to ease pain at the site of the injection. The ingredients may be supplied either separately or mixed together in unit dosage form, for example, as a dry lyophilized powder or water free concentrate in a hermetically sealed container such as an ampoule or sachette indicating the quantity of active agent. Where the composition is to be administered by infusion, it may be dispensed with an infusion bottle containing sterile pharmaceutical grade water or saline. Where the composition is administered by injection, an ampoule of sterile water for injection or saline may be provided so that the ingredients may be mixed prior to administration.

### BRIEF DESCRIPTION OF THE DRAWINGS

FIG. 1 shows the levels of repression of Renilla signal normalized to Firefly as percent of empty vector. n,N=2-3,4-6, mean±SEM. The most potent anti-sense oligonucleotides are SEQ ID NO.: 8, 12, 16, 19 and 20.

FIG. 2 shows the dose-response curves and the IC50 values of the five miR-27b antisense oligonucleotides measured in PC-12 cells. Dose-response curves and IC50 values, n,N=1,2, both technical replicates are shown, 3-parameter non-linear curve fit.

FIG. 3 shows IC50 values for Seq ID 8, 12, 16, 19 and 20, measured by derepression of Renilla luciferase activity in U-87 mg cells, n,N=2, 4-6, all biological replicates are depicted. IC50 curves were fitted, and potency calculated using least squares regression with log(inhibitor) vs. a three-parameter response.

FIG. 4 shows increase in expression of miR-27b direct (nrf2) and downstream (hmox1, nqo1) target mRNAs after transfection of antimiR-27b oligonucleotides into PC-12 Adh cells; n,N=3,6; mean±SEM, all technical replicates are depicted. qPCR results were analysed using the ΔΔCt method using a scrambled oligonucleotide for normalisation. (The oligonucleotide defined as Seq ID 20 used in this experiment was inosine substituted on one guanine to make it correspond to Seq ID 22).

FIG. 5 shows the potency of Seq ID 20 and inosine-substituted Seq ID 22. A: Derepression of Renilla luciferase activity after transfection with 0.2, 1 and 5 nM antimiRs into PC-12 Adh cells. n,N=1,2; mean±SEM. All technical replicates are depicted. B: Dose-response curves and the IC50 values of Seq ID 20 and Seq ID 22. Seq ID 22 is inosine substituted on one guanine but otherwise identical to Seq ID 20. Dose-response curves and IC50 values, n,N=1,3, mean, 3-parameter non-linear curve fit.

### EXAMPLES

### Example 1: Cell Culture

In vitro modelling of the effects on miRNA of antisense oligonucleotides is commonly done in mammalian cell lines.

The adherent rat pheochromocytoma cell line PC-12 Adh (ECACC no. 88022401) was purchased from ATCC (ATCC cat. no. CRL-1721.1™) and grown in Corning® CeIIBIND® Surface cell culture flasks (Sigma-Aldrich cat. no. CLS3290) in Ham's F-12K (Kaighn's) medium (ThermoFischer Scientific cat. no. 21127022) supplemented with 2.5% heat-inactivated fetal bovine serum (Sigma-Aldrich cat. no F4135-500 ml), 15% heat-inactivated horse serum (Sigma-Aldrich cat. no. H1385-500 ml and 1% penicillin/streptomycin (Sigma-Aldrich cat. no. P4333-100 ml). The cells were kept in in a humidified 5% C02 incubator at 37° C. and passaged twice a week.

### Example 2: Luciferase Reporter Assays in Cultured Cell Lines

A simple and very sensitive approach involves construction of a miRNA reporter plasmid that carries a single perfect match miRNA binding site in the 3′ UTR of a reporter gene, such as luciferase. This method has been extensively used in cultured cells to validate miRNA inhibition and also to compare the potency of different antimiR designs.

The miR-27b reporter was generated by cloning annealed oligonucleotides corresponding to single perfect-match target site for human miR-27b into the 3′ UTR of the Renilla luciferase gene in the dual-luciferase psiCHECK2 plasmid (Promega).

For luciferase assays, PC-12 adh cells were seeded in 96-well Corning® CeIIBIND® Surface cell culture microwell plates (Sigma-Aldrich cat. no. CLS3330) at a density of 25,000 cells per well the day before transfection. The cells were transfected using lipofectamine 2000 (ThermoFischer Scientific cat. no. 11668-019) at a final concentration of 0.5 μL/well in Opti-MEM™ I Reduced Serum Medium, GlutaMAX™ Supplement (ThermoFischer Scientific cat. no. 51985026). A library of 17 antisense oligonucleotides was screened using the lucirease reporter assays by co-transfecting each antimiR-27b with the luciferase reporter plasmid and the miR-27b mimic in final concentrations of 0.2 nM, 1 nM, 5 nM. A scrambled sequence oligonucleotide, a vector containing no miRNA match site and a mock transfection were included as controls. All samples were run in technical duplicates. After 4 hours the cells were washed in Opti-MEM™ medium and fresh complete cell culture medium was added to the wells.

24 hours after transfection the luciferase assay was conducted using Dual-Glo@ Luciferase Assay System (Promega cat. no. E2920) as per manufacturer's instructions. The amount of luminescence was determined on a plate reader (VarioSkan Lux, ThermoFischer Scientific) after 30 minutes incubation of reagents in the plates.

The results were analysed by subtraction of background luminescence and then normalizing Renilla signal to the constitutive Firefly signal. The average of the two technical duplicates were then normalized to empty vector and expressed as percentage. The results were visualized in in Graphpad Prism (version 9.0.2, GraphPad Software).

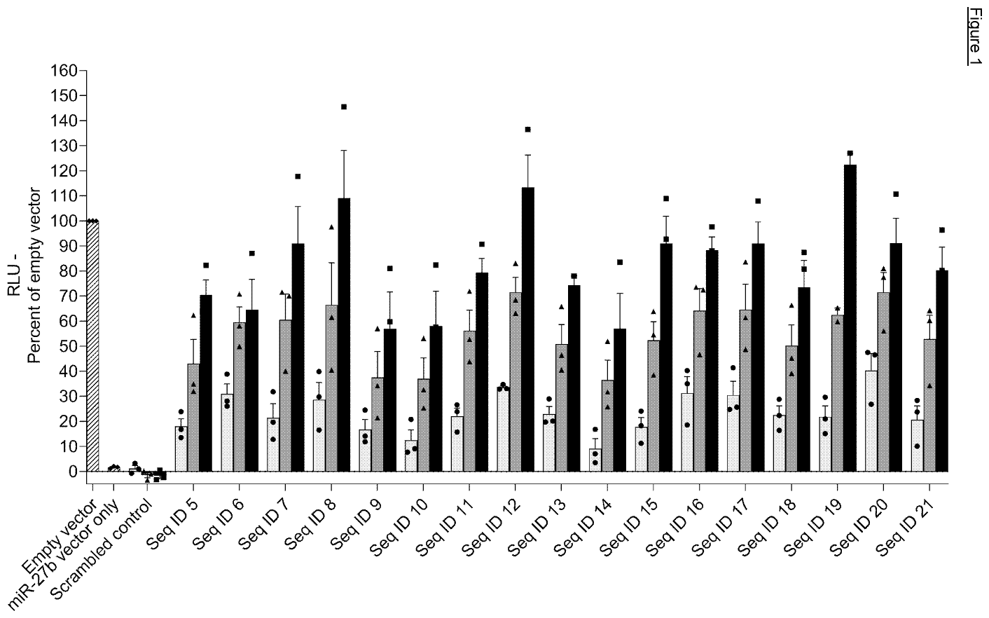

The levels of derepression of Renilla luciferase activity normalized to Firefly luciferase activity for all 17 antisense oligonucleotides are shown in FIG. 1.

From the full library of antimiR-27b antisense oligonucleotides the five most potent antimiR-27b molecules were chosen for further analyses and IC50 determinations.

### Example 3: Determination of IC50 for AntimiR-27b Oligonucleotides in Cultured Cell Lines

To determine the potency of antisense oligonucleotides in inhibiting miR-27b, IC50 determinations were conducted. The luciferase assays were carried out as described in example 2. The antisense oligonucleotides were compared to miR-27b antagomir from Xu et al (Oncotarget. 2017 Sep. 19; 8(41): 70669-70684). For the determination of IC50 values, the cells were transfected with a wide range of antimiR-27b concentrations ranging from 80 nM in 2-fold dilutions to 0.0049 nM. The Renilla luciferase activity was normalized to Firefly luciferase activity and plotted against log(M) in Graphpad Prism (version 9.0.2, GraphPad Software). The dose-response curves were fitted using 3-parameter non-linear fit and IC50 values calculated in nM. It was not feasible to determine IC50 value for the antagomiR control compound due to the low response across the selected concentrations.

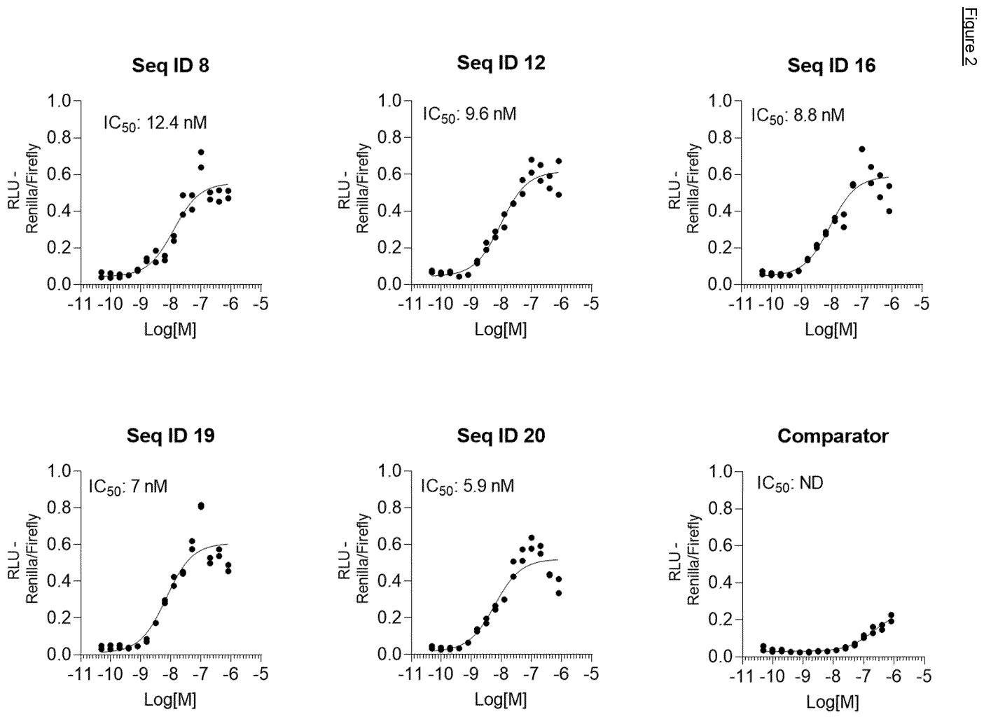

FIG. 2 shows the dose-response curves and the IC50 values of the five antimiR-27b oligonucleotides.

### Example 4: IC50 Determination in Cultured U-87 Mg Cells

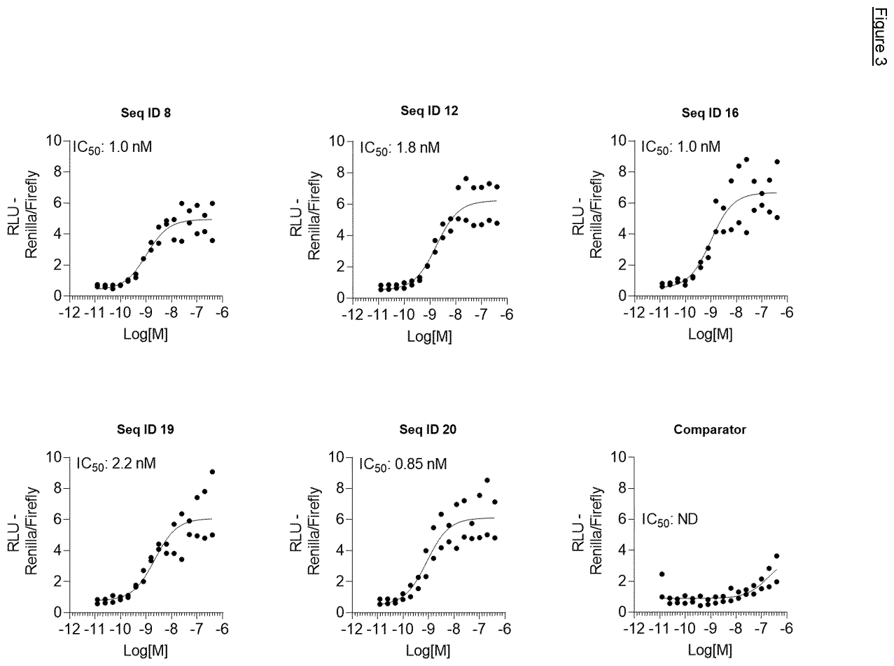

The IC50 curves in U-87 Mg cells were done as in the PC-12 Adh cells described in above examples, except that the amount of Lipofectamine 2000 was 0.4 μL per well and the transfections were done in 96-well Costar black plates (cat. no: 3603, Corning World, Corning, NY, USA). FIG. 3 shows the dose response curves and the IC50 values of five selected antimiR-27b oligonucleotides (seq id no's: 8, 12, 16, 19 and 20).

Example 5 shows miR-27b target mRNA derepression in a cultured PC-12 Adh cell line. Since miRNAs negatively regulate levels of their target mRNAs, the functional effects of miR-27b inhibition by antimiR oligonucleotides can be measured by a subsequent upregulation of target mRNAs. The principal target of miR-27b is the transcription factor Nrf2; responsible for the upregulation of antioxidant and detoxifying factors such as Hmox1 and Nqo1. Upregulation in these three markers signify not just a functional effect on Nrf2 levels but also shows activation of the down-stream molecular pathways regulated by Nrf2.

The PC-12 Adh cells were transfected as described in the examples above with the exception that the cells were seeded in 12-well CellBind plates (cat. no: CLS3336, Corning World, Corning, NY, USA) at 3×105 cells/well, using 6 μL Lipofectamine2000 per well and no luciferase reporter was used. A FAM-labelled oligonucleotide was transfected in a separate well to confirm transfection efficiency by examination by direct microscopy. Forty-eight hours after transfection, RNA extraction was conducted using the miRNeasy mini kit (cat. no: 217004, Qiagen, Hilden, Germany) as per manufacturer's instructions. The RNA was stored at −80-C until further analysis. Reverse transcription was conducted using Superscript IV reverse transcriptase (cat. no: 18090010, Thermo Fischer Scientific, Waltham, MA, USA) as per manufacturer's instructions, including gDNA removal by ezDNase™ (cat. no: 11766051, Thermo Fischer Scientific, Waltham, MA, USA) and using a random hexamer primer (cat. no: SO142, Thermo Fischer Scientific, Waltham, MA, USA). The qPCR was done on a QuantStudio 6 Flex (Applied Biosystems, Waltham, MA, USA) using Taqman assays (Table 2) synthesized by Integrated DNA Technologies (Newark, NJ, USA) and TaqMan™ Universal Master Mix II, no UNG (cat. no: 4440040, Thermo Fischer Scientific, Waltham, MA, USA) as per manufacturer's instructions. All qPCR assays were designed to be exon-spanning and specificity was confirmed by blast of the primers and the efficiency of primers was tested using a five-fold dilution series. Hprt1 was used as a house-keeping gene. All qPCR results were analysed using the ΔΔCt method (Livak K J, Schmittgen T D. Analysis of Relative Gene Expression Data Using Real-Time Quantitative PCR and the 2-ΔΔCT Method. Methods. 2001; 25(4):402-408) using a scrambled oligonucleotide for normalisation.

TABLE 2

| | Forward | Reverse | | |
|---|---|---|---|---|
| Gene | primer | primer | Probe | Cat.no: |
| Nrf2 | CACTCTG | GAATGTG | /56-FAM/ATTTCCGAG/ | Custom- |
| | TGGAGTC | TTGGCTG | ZEN/TCACTGAACCCAGG | made |
| | TTCCATT | TGCTTTA | C/3IABkFQ/ | |
| | T | G | | |
| Hmox1 | GATGGCC | AGCTCCT | /5HEX/AGAGCGAAA/ | Custom- |
| | TCCTTGT | CAGGGAA | ZEN/CAAGCAGAACCCAG | made |
| | ACCATAT | GTAGAG | T/3IABkFQ/ | |
| | C | | | |
| Nqo1 | GCTGCAG | ACATGGT | /56-FAM/AGGCTGGTT/ | Custom- |
| | ACCTGGT | GGCATAC | ZEN/TGAGAGAGTGCTTG | made |
| | GATATT | GTGTAG | T/3IABkFQ/ | |
| Hprt1 | GGAGAAC | TGTGAAG | /56-FAM/TGTTGACCC/ | Rn.PT. |
| | AATTCTG | TTCCCCA | ZEN/ACCAGCAGTTCAG | 39a. |
| | GGTTTGA | TAAGGC | T/3IABkFQ/ | 22214832 |
| | TC | | | |

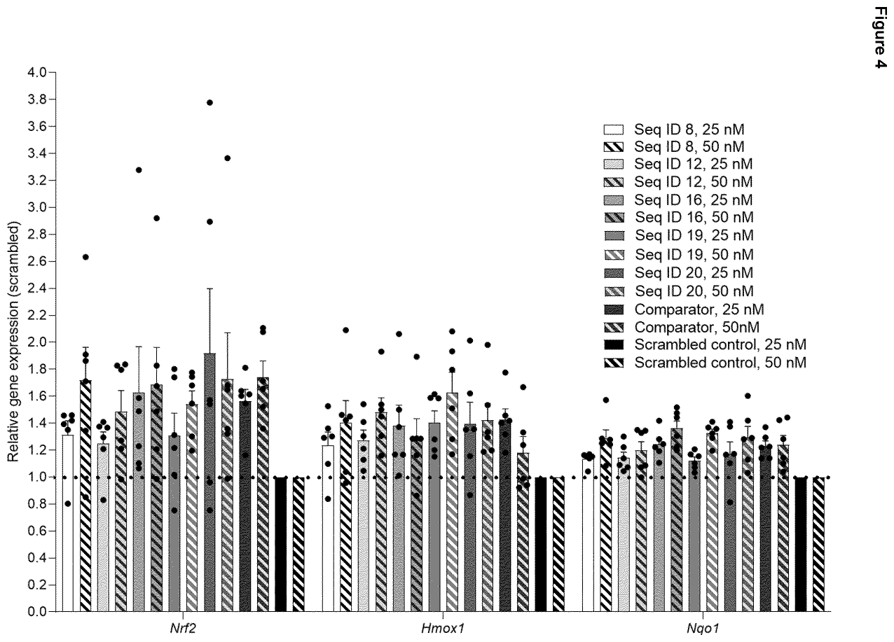

The bar diagram in FIG. 4 shows the effect of antimiRs (Seq ID 8, 12, 16, 19 and 20) on miR-27b target gene derepression (Nrf2, Hmox1 and Nqo1). The oligonucleotide defined as Seq ID 20 used in this experiment was inosine substituted on one guanine to make it correspond to Seq ID 22.

### Example 6 Shows the Assessment of the Potency of Seq ID 20 and Seq ID 22

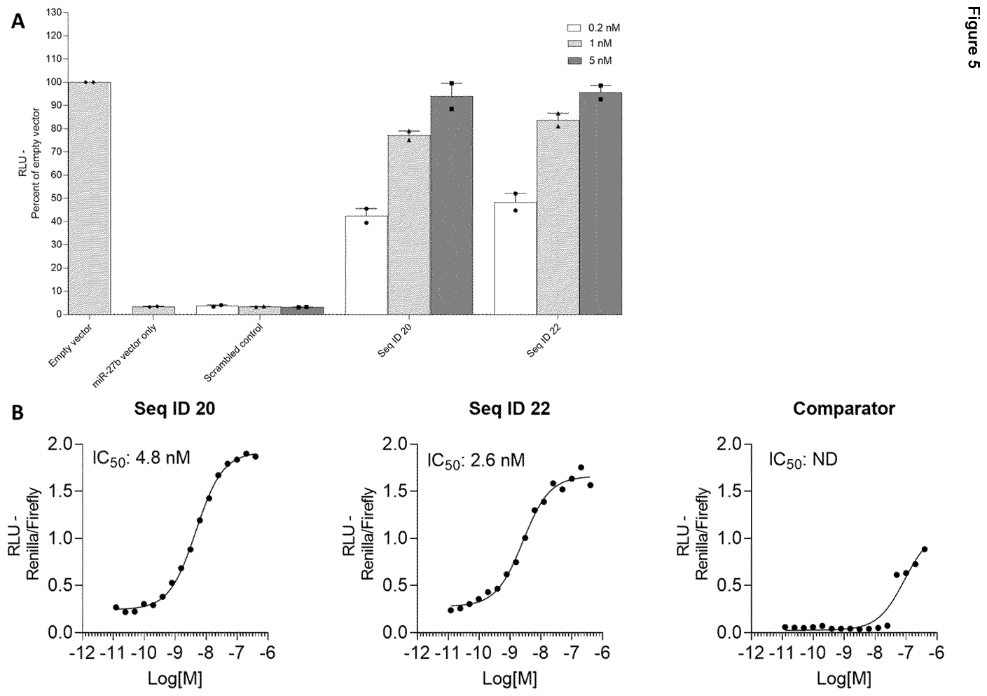

The transfection and luciferase assay were done as described in example 2 and 3, except that the cells were seeded in clear-bottom, white 96-well plates (cat. no 3610, Corning) pretreated with collagen (Sigma-Aldrich cat. no. C8919) and for the IC50 experiment three technical replicates were used and no background subtraction conducted. The results of the dose-response experiment and IC50 experiment are shown in FIG. 5 A and B, respectively.

### Embodiments

1) An antisense oligonucleotide complementary to miR-27b (SEQ ID NO: 1 or 2) comprising a sequence of 18-19 nucleotides in length, wherein the antisense oligonucleotide is a mixmer having from seven to 14, such as from 10-13 affinity-enhancing nucleotide analogues and does not contain a stretch of more than three contiguous DNA nucleotides, and wherein said antisense oligonucleotide comprises one to 18 phosphorothioate internucleoside linkages.

2) The antisense oligonucleotide according to Embodiment 1, wherein the antisense oligonucleotide is complementary to SEQ ID NO: 3.

3) The antisense oligonucleotide according to embodiment 1 or 2, which comprises SEQ ID NO: 4.

4) The antisense oligonucleotide according to any one of embodiments 1 to 3, wherein the antisense oligonucleotide is 18 or 19 nucleotides in length, comprises SEQ ID NO: 4 and is a LNA/DNA mixmer.

5) The antisense oligonucleotide according to any one of embodiments 1 to 4, wherein the antisense oligonucleotide is 18 or 19 nucleotides in length, comprises SEQ ID NO: 4 and is a LNA/DNA mixmer having between 50 and 70% LNA, such as between 52 and 68% LNA, such as at least 50% LNA, such as at least 52% LNA.

6) The antisense oligonucleotide according to any one of embodiments 1 to 5, wherein the two terminal nucleotides in each end are LNA.

7) The antisense oligonucleotide according to any one of embodiments 1 to 6, wherein the LNA is Beta-D-Oxy LNA.

8) The antisense oligonucleotide according to any one of embodiments 1 to 7, wherein all the internucleoside bonds are phosphorothioate bonds.

9) The antisense oligonucleotide according to any one of embodiments 1 to 8, wherein the antisense oligonucleotide is anyone of SEQ ID NO's 5-22.

10) The antisense oligonucleotide according to embodiment 9, wherein all LNA's are beta-D-oxy LNA, all LNA cytosines are 5-methylcytosine, and all internucleoside bonds are phosphorothioate bonds.

11) The antisense oligonucleotide according to anyone of embodiments 1 to 10, wherein the LNA/DNA mixmer further comprises one or more nucleosides that are anyone of tricyclo-DNA, 2′-Fluoro, 2′-O-methyl, 2′methoxyethyl (2′MOE), 2′ cyclic ethyl (cET), UNA, 2′fluoro and Conformationally Restricted Nucleoside (CRN).

12) The antisense oligonucleotide according to any one of embodiments 1 to 11, for use as a medicament.

13) A miR-27b inhibitory composition comprising the antisense oligonucleotide according to anyone of embodiments 1 to 12.

14) The composition according to embodiment 13, for use in inducing the Nrf-2/ARE pathway in a mammal, such as in a human.

15) The antisense oligonucleotide for use as a medicament according to embodiment 12, or the composition according to embodiment 13 or 14, wherein the antisense oligonucleotide is anyone of SEQ ID NO's: 5-22).

16) The use or composition according to any of embodiments 12, 13, 14 or 15, wherein the use is for the treatment, alleviation, pre-emptive treatment or prophylaxis of a miR-27b related disease where modification of miR-27b activity, or induction of the Nrf-2/ARE pathway is beneficial.

17) The use or composition according to embodiment 12, 13, 14, 15 or 16, wherein the use is for the treatment, alleviation, pre-emptive treatment or prophylaxis of a miR-27b related disease of the CNS or PNS.

18) The use according to embodiment 17, wherein the use is for treatment, alleviation, pre-emptive treatment or prophylaxis of a neurological disorder.

19) The use according to embodiment 18, wherein the use is for treatment, alleviation, pre-emptive treatment or prophylaxis of a neurodegenerative disorder.

20) The use according to embodiment 19, wherein the use is for treatment, alleviation, pre-emptive treatment or prophylaxis of a neurodevelopmental disorder, a genetic disorder and/or a genetic neurodevelopmental disorder.

21) The use according to anyone of embodiments 12 to 19, wherein the use is for treatment, alleviation, pre-emptive treatment or prophylaxis of epilepsy.

22) The use according to embodiment 21, wherein the use is for treatment, alleviation, pre-emptive treatment or prophylaxis of drug resistant epilepsy.

23) The use according to embodiment 21 or 22, wherein the use is for treatment, alleviation, pre-emptive treatment or prophylaxis of seizures in epilepsy.

24) The use according to embodiment 21 to 23, wherein the use is for treatment, alleviation, pre-emptive treatment or prophylaxis of spontaneous seizures in epilepsy.

25) The use according to embodiment 21 to 24, wherein the use is for treatment, alleviation, pre-emptive treatment or prophylaxis of therapy resistant seizures.

26) The use according to embodiment 21 to 25, wherein said epilepsy is a focal epilepsy, preferably wherein said focal epilepsy is focused in the frontal lobe, the parietal lobe, the occipital lobe or the temporal lobe.

27) The use according to embodiment 21 to 25, wherein said epilepsy is a generalised epilepsy, preferably wherein said generalised epilepsy is selected among absences, myoclonic seizures, tonic-clonic seizures, tonic seizures, atonic seizures, clonic seizures and spasms.

28) The use according to embodiment 21 to 27, wherein said epilepsy is status epilepticus.

29) The use according to embodiment 21 to 28, wherein said epilepsy is selected among autosomal dominant nocturnal frontal lobe epilepsy, continuous spike-and-waves during slow sleep, Dravet syndrome, epilepsy developed after apoplexy, epileptic encephalopathy, Gelastic epilepsy, absences, benign neonatal seizures, Jeavons syndrome, Juvenile myoclonic epilepsy, Landau-Kleffner Syndrom, Lennox-Gastaut syndrome, Mesial temporal lobe epilepsy, myoclonic astatic epilepsy, Ohtahara Syndrom, Panayiotopoulos syndrome, PCDH19 syndrom, benign childhood epilepsy with centrotemporal spikes, Sturge-Weber syndrome, symptomatic focal epilepsy, transient epileptic amnesia and West syndrome.

30) The use according to embodiment 21 to 29 wherein said epilepsy is present together with a comorbidity selected among a psychiatric disorder, a cognitive disorder, a sleep disorder, a cardiovascular disorder, a respiratory disorder, an inflammatory disorder, a psychiatric disorder, anxiety, pain, cognitive impairment, depression, dementia, headache, migraine, heart disease, ulcers, peptic ulcers, arthritis and osteoporosis.

31) The use according to embodiments 16 to 29, wherein the use is for prevention or prophylaxis or alleviation or treatment of neuronal damage.

32) The use according to embodiment 31, wherein the use is for treatment, alleviation, pre-emptive treatment or prophylaxis of hippocampal damage.

33) The use according to embodiment 17, wherein the use is for treatment, alleviation, pre-emptive treatment or prophylaxis of oxidative stress, inflammation and/or apoptosis.

34) The use according to embodiment 17, wherein the use is for treatment, alleviation, pre-emptive treatment or prophylaxis of intracerebral hemorrhage-induced brain injury, ischemic stroke, hemorrhagic stroke or stroke.

35) The use according to embodiment 17 to 20, wherein the use is for treatment, alleviation, pre-emptive treatment or prophylaxis of an autoimmune disease, a memory disorder, hippocampal sclerosis, Parkinsons Disease, a demyelinating disease, multiple sclerosis, spinal cord injury, acute spinal cord injury, amyotrophic lateral sclerosis, Progressive bulbar palsy, Progressive muscular atrophy, Primary lateral sclerosis, ataxia, bell's palsy, a hereditary neurological disease, Charcot-Marie-Tooth, a headache, Horton's headache, migraine, pick's disease, progressive supranuclear palsy, multi-system degeneration, motor neuron diseases, Huntington's disease, prion disease, Creutzfeldt-Jakob disease, corticobasal degeneration, aphasia, primary progressive aphasia or symptoms or effects thereof.

36) The use according to embodiment 17 to 19 or 35, wherein the use is for treatment, alleviation, pre-emptive treatment or prophylaxis of dementia.

37) The use according to embodiment 36, wherein said dementia is selected among Alzheimer disease, vascular dementia, frontotemporal dementia and Lewy bodies dementia.

38) The use according to embodiment 12 or any of embodiments 14 to 19, wherein the use is for treatment, alleviation, pre-emptive treatment or prophylaxis of pain, such as pain associated with osteoarthritis.

39) The use according to any of embodiments 12 to 19, wherein the use is for treatment, alleviation, pre-emptive treatment or prophylaxis of a psychiatric disease wherein modulation of miR-27b is beneficial.

40) The use according to embodiment 39, wherein the use is for treatment, alleviation, pre-emptive treatment or prophylaxis of schizophrenia, depression, bipolar disorder, attention deficit hyperactivity disorder, autism, anxiety or Tourette.

41) The use according to embodiment 12 to 16, wherein the use is for treatment, alleviation, pre-emptive treatment or prophylaxis of an angiogenesis related disease.

42) The use according to any one of embodiment 12 to 16 or 41, wherein the use is for treatment alleviation, pre-emptive treatment or prophylaxis of a cancer.

43) The use according to embodiment 42, wherein said cancer is a cancer in the nerve system, preferably glioma.

44) The use according embodiment 42, wherein said cancer is a cancer in the skin, preferably melanoma.

45) The use according to embodiment 42, wherein said cancer is a head or neck cancer.

46) The use according to embodiment 42, wherein said cancer is a squamous cell carcinoma, preferably tongue squamous cell carcinoma or oral squamous cell carcinoma.

47) The use according to embodiment 42, wherein said cancer is a hematologic cancer, preferably myeloma or lymphoma, more preferably diffuse large B-cell lymphoma.

48) The use according to embodiment 42, wherein said cancer is a breast cancer, preferably triple negative breast cancer.

49) The use according to embodiment 42, wherein said cancer is a thyroid cancer, preferably anaplastic thyroid cancer.

50) The use according to embodiment 42, wherein said cancer is a liver cancer, preferably hepatocellular carcinoma.

51) The use according to embodiment 42, wherein said cancer is selected from the group of gastric cancer, cervical cancer, endometrial cancer, hemangioma, lung cancer, pancreatic cancer, bladder cancer, prostate cancer and colorectal cancer, such as migration and invasion in colorectal cancer.

52) The use according to embodiment 42 to 51, wherein said cancer is a cancer metastasis.

53) The use according to any one of embodiments 12 to 16 or 20, wherein the antisense oligonucleotide is for use in treating Prader-Willis Syndrome or Anglemans Syndrome.

54) The use according to embodiment 12 or any one of 14 to 16 wherein the use is for treatment, alleviation, pre-emptive treatment or prophylaxis of an arthritis.

55) The use according to embodiment 54, wherein said arthritis is osteoarthritis.

56) The use according to embodiment 12 or any one of 14 to 17, wherein the use is for treatment, alleviation, pre-emptive treatment or prophylaxis of a cardiovascular disorder.

57) The use according to embodiment 56, wherein said cardiovascular disorder is selected among atherosclerosis, peripheral artery disease, postoperative atrial fibrillation, heart failure and chronic heart failure, intracerebral haemorrhage-induced brain injury or stroke.

58) The use according to embodiment 12 or any one of 14 to 16, wherein the use is for treatment, alleviation, pre-emptive treatment or prophylaxis of a liver disorder.

59) The use according to embodiment 58Error! Reference source not found, wherein said liver disorder is selected among non-alcoholic fatty liver, fatty liver, fatty liver fibrosis, liver fibrosis and hepatoma.

60) The use according to embodiment 12 or any one 14 to 16, wherein said use is for treatment, alleviation, pre-emptive treatment or prophylaxis of a pulmonary disorder, such as pulmonary sarcoidosis.

61) The use according to embodiment 12 or any one of 14 to 16, wherein said use is for treatment, alleviation, pre-emptive treatment or prophylaxis of an autoimmune disease, a granulomatous disease, a connective tissue disease or sarcoidosis.

62) The use according to any one of embodiment 12 or any one of 14 to 17, wherein the use is for treatment, alleviation, pre-emptive treatment or prophylaxis of an infection.

63) The use according to embodiment 62, wherein said infection is selected among sepsis, meningitis and encephalitis.

64) The use according to embodiment 62, wherein said infection is a herpes virus infection.

65) The use according to embodiment 62, wherein said infection is a human papilloma virus infection.

66) The use according to embodiment 64, wherein said herpes virus infection is selected between a herpes simplex virus infection and a Cytomegalovirus infection.

67) The use according to embodiment 12 or any one of 14-17 or 41, wherein said use is for treatment, alleviation, pre-emptive treatment or prophylaxis of a disorder of the retina.

68) The use according to embodiment 67, wherein said disorder of the retina is selected among retinopathy, diabetic retinopathy and age-related macular degeneration (AMD).

69) The use according to embodiment 12 or any one of 14 to 16, wherein said use is for treatment, alleviation, pre-emptive treatment or prophylaxis of a metabolic disorder.

70) The use according to embodiment 69, wherein said metabolic disorder is diabetes, preferably type 2 diabetes.

71) The use according to embodiment 12 or any one of 14 to 17, wherein said use is for treatment, alleviation, pre-emptive treatment or prophylaxis of a genetic disorder, preferably neurofibromatosis.

72) The use according to anyone of embodiments 12 to 71, wherein the antisense oligonucleotide is for use in combination with another therapy.

73) The use according to embodiment 72, wherein said other therapy is an anti miR-134 antisense oligonucleotide.

74) The use according to embodiment 72, wherein said other therapy is an adenosine kinase inhibitor.

75) The use according to embodiment 72, wherein said other therapy induces the Nrf-2/ARE pathway in a mammal, such as in a human.

76) The use according to embodiment 72, wherein said therapy is one or more of an anti miR-134 antisense onligonucleotide, an adenosine kinase inhibitor and a therapy inducing the Nrf-2/ARE pathway.

77) The use according to anyone of embodiment 12 to 71, wherein the antisense oligonucleotide of the invention is the sole active pharmaceutical ingredient.

78) A pharmaceutical composition comprising the antisense oligonucleotide according to anyone of embodiments 1 to 12 and a pharmaceutically acceptable carrier.

79) A pharmaceutical composition comprising the antisense oligonucleotide according to anyone of embodiment 1 to 12, wherein said antimiR27b oligonucleotide is the sole active pharmaceutical ingredient.

80) The pharmaceutical composition according to any one of embodiment 78 to 80, wherein the composition is for use according to any one embodiments 12 to 77.

81) The pharmaceutical composition according to embodiments 78 to 80, wherein the composition is for administration by subcutaneous administration, intravenous administration, parenteral administration, nasal administration, pulmonary administration, rectal administration, vaginal administration, intrauterine administration, Intraurethral administration, administration to the eye, administration to the ear, cutaneous administration, intradermal administration, intramuscular administration, intraperitoneal administration, epidural administration, intraventricular administration, intracerebral, intrathecal administration or oral administration or for administration directly into the brain or cerebrospinal fluid, or wherein said composition is administered as an implant.

82) The pharmaceutical composition according to embodiment 78 to 81, wherein said composition is administrated in a pump, preferably wherein said pump is a mini-osmotic pump.

83) The pharmaceutical composition according to embodiment 78 to 82, wherein said composition is for intraventricular administration facilitated by an intraventricular catheter, preferably wherein said catheter is attached to a reservoir, preferably wherein said reservoir is an Ommaya reservoir.

84) The pharmaceutical composition according to embodiment 81 to 83, wherein said composition is administrated with an interval of 1 day, 2 days, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or preferably 120 days.

85) The pharmaceutical composition according to embodiment 81 to 83, wherein said composition is administrated with an interval of between 1-200 days, 10-190 days, 20-180 days, 30-170 days, 40-160 days, 50-150 days, 60-140 days, 70-130 days, 80-120 days, 90-110 days or preferably about 100 days.

86) The antisense oligonucleotide according to any one of embodiments 1 to 12 or the composition according to embodiment 13 for use in a method of treating the diseases according to any one of embodiments 12 to 77.

87) A method for the treatment of the diseases according to any one of embodiments 12 to 77 by use of the antisense oligonucleotide according to any one of embodiments 1 to 12r the composition according to embodiment 13 or 14.

88) The use according to any one of embodiments 12 to 77, or pharmaceutical composition according to any one of embodiments 78 to 85, or the method according to embodiment 86 or 87, wherein the treatment is anyone of preventive, curative or disease modifying.

89) A method of diagnosing a disease according to any one of embodiment 12 to 77 by use of the antisense oligonucleotide according to any one of embodiments 1 to 12 or the composition according to embodiment 13 or 14.

1. An antisense oligonucleotide comprising a sequence of 18-19 nucleotides in length complementary to miR-27b, wherein the antisense oligonucleotide is a mixmer having from seven to 14 affinity-enhancing nucleotide analogues and does not contain a stretch of more than three contiguous DNA nucleotides, and wherein said antisense oligonucleotide comprises one to 18 phosphorothioate internucleoside linkages.

2-15. (canceled)

16. The antisense oligonucleotide according to claim 1, wherein the antisense oligonucleotide is complementary to the sequence set forth by SEQ ID NO: 3.

17. The antisense oligonucleotide according to claim 1, which comprises the sequence set forth by SEQ ID NO: 4.

18. The antisense oligonucleotide according to claim 1, wherein the antisense oligonucleotide is 18 or 19 nucleotides in length, comprises the sequence set forth by SEQ ID NO: 4 and is a LNA/DNA mixmer.

19. The antisense oligonucleotide according to claim 1, wherein the antisense oligonucleotide is 18 or 19 nucleotides in length, comprises the sequence set forth by SEQ ID NO: 4 and, wherein between 50 and 70% of the nucleosides of said mixmer is LNA.

20. The antisense oligonucleotide according to claim 1, wherein the two terminal nucleotides in each end are LNA.

21. The antisense oligonucleotide according to claim 1, wherein the LNA is Beta-D-Oxy LNA and LNA cytosines are 5-methylcytosine.

22. The antisense oligonucleotide according to claim 1, wherein all the internucleoside bonds are phosphorothioate bonds.

23. The antisense oligonucleotide according to claim 1, wherein the antisense oligonucleotide is anyone of the sequences set forth by SEQ ID NO's 22, 21, 20, 19, 18, 17, 16, 15, 14, 13, 12, 11, 10, 9, 8, 7, 6, 5, 4, 3, 2, or 1.

24. The antisense oligonucleotide according to claim 23, wherein the antisense oligonucleotide is anyone of:
(SEQ ID NO 22)
5′ AGAacTTaiCcACTgtGA 3′
(SEQ ID NO 20)
5′ AGAacTTagCcACTgtGA 3′
(SEQ ID NO 19)
5′ CAgaaCTtaGccACtgTGA 3′
(SEQ ID NO 16)
5′ AGaActTagCcaCTgTGA 3′
(SEQ ID NO 12)
5′ AGaaCTtAGcCaCtgTGA 3′
or
(SEQ ID NO 8)
5′ AGaActTAgcCaCTGtGA 3′
wherein capital letters are LNA, small letters are DNA, capital C denotes LNA 5-methylcytosine, LNA is beta-D-oxy LNA, “i” is inosine and all internucleoside bonds are phosphorothioate bonds.

25. The antisense oligonucleotide according to claim 1, wherein the mixmer is a LNA/DNA mixmer further comprising one or more nucleosides that are anyone of tricyclo-DNA, 2′-Fluoro, 2′-0-methyl, 2′methoxyethyl (2′MOE), 2′ cyclic ethyl (cET), UNA, 2′fluoro or Conformationally Restricted Nucleoside (CRN).

26. A pharmaceutical composition comprising an effective dosage of the antisense oligonucleotides of claim 1.

27. A method for the treatment, alleviation, amelioration, pre-emptive treatment or prophylaxis treatment of a miR-27b related disease of the CNS or PNS, said method comprising administrating to a subject in the need thereof an antisense oligonucleotide comprising a sequence of 18-19 nucleotides in length complementary to miR-27b, wherein the antisense oligonucleotide is a mixmer having from seven to 14 affinity-enhancing nucleotide analogues and does not contain a stretch of more than three contiguous DNA nucleotides, and wherein said antisense oligonucleotide comprises one to 18 phosphorothioate internucleoside linkages.

28. The method according to claim 27, wherein the disease of the CNS or PNS is a neurological disorder.

29. The method according to claim 27, wherein the disease of the CNS or PNS is epilepsy.
